(19) United States
(12) Patent Application Publication
(10) Pub. No.: US 2008/0206145 A1
(43) Pub. Date: Aug. 28, 2008

(54) Gastro-Retentive Diagnostic Assemblies

(76) Inventors: Michel Afargan, Ranana (IL); David Kirmayer, Ma'ale Edomim (IL); Noa Lapidot, Mevasseret Zion (IL); Michael Friedman, Jerusalem (IL); Amnon Hoffman, Jerusalem (IL)

Correspondence Address: NATH & ASSOCIATES, 112 South West Street, Alexandria, VA, 22314, US

(21) Appl. No.: 10/568,144

(22) PCT Filed: Jan. 5, 2006
(86) PCT No.: PCT/IL2006/000017

§ 371 Date: Feb. 13, 2006

(30) Foreign Application Priority Data
Jan. 6, 2005 (IL) 166183

(51) Int. Cl. A61K 49/06; A61K 49/00; A61P 1/00; A61K 49/04

(52) U.S. Cl. 424/9.3; 424/9.1; 424/9.4; 424/9.5

(57) ABSTRACT

The present invention provides a gastro-retentive diagnostic assembly (GRDA) for use in determining a condition of a subject's gastrointestinal tract (GI tract), comprising a folded single or multi-layered device comprising a diagnostic utility, such as a contrasting agent, the device prior to folding being essentially planar, and included in a delivery system for oral intake, the delivery system being adapted to release the device once in the stomach, whereupon release said device unfolds into an unfolded shape that results in the retention of the device in the stomach. Further provided are methods of determining a condition of a subject's GI tract by the use of the GRDA of the invention, as well as uses of a single or multi-layered device comprising a diagnostic utility when in a folded or unfolded shape, and method of preparing the GRDA of the invention.

### FIELD OF THE INVENTION

The invention relates to the field of diagnostics and more specifically to imaging of the gastrointestinal tract.

### LIST OF PRIOR ART

The following is a list of prior art, which is considered to be pertinent for describing the state of the art in the field of the invention.
(1) Steingoetter A, Weishaupt D, Kunz P, Meder K, Lengsfeld H, Thumshirn M, Boesiger P, Fried M, Schwizer W. Magnetic resonance imaging for the in vivo evaluation of gastric-retentive tablets. Pharm Res, 20:2001-7 (2003).
(2) Steingoetter A, Kunz P, Weishaupt D, Meder K, Lengsfeld H, Thumshirn M, Boesiger P. Fried M, Schwizer W. Analysis of the meal-dependent intragastric performance of a gastric-retentive tablet assessed by magnetic resonance imaging. Aliment. Pharmacol. Ther, 18:713-20 (2003).
(3) Shalaby W S, Blevins W E, Park K. Use of ultrasound imaging and fluoroscopic imaging to study gastric retention of enzyme-digestible hydrogels. Biomaterials, 13:289-96 (1992).
(4) U.S. Pat. No. 6,685,962

### BACKGROUND OF THE INVENTION

Diagnostic techniques of internal organs play an important role in modern medical practice.

There are numerous approaches and diagnostic procedures used for detecting conditions of the GI tract. These include, for example, laboratory fecal occult blood test and stool culture, Imaging test using barium beefsteak meal, Colorectal transit study, Computed tomography scan (CT or CAT scan), Defecography, Lower GI series (also called barium enema), Magnetic Resonance Imaging (MRI), Oropharyngeal motility (swallowing) study, Radioisotope gastric-emptying scan, Ultrasound, Upper GI series (also called barium swallow), Endoscopic procedures (Colonoscopy, Endoscopic retrograde cholangiopancreatography, Esophagogastroduodenoscopy, Sigmoidoscopy), Exhaled hydrogen test. Anorectal manometry. Esdphageal manoinetry, pH monitoring, and Gastric manometry.

Reports on the utilization of MRI for the evaluation of the gastrointestinal tract have already been described [Chou, C. K. et al. (1994a) MRI manifestations of gastrointestinal wall thickening. Abdom Imaging; 19:389-394; Chou, C. K. et al. (1994b) MRI manifestations of gastrointestinal lymphoma. Abdom Imaging; 19:495-500; Ha, H. K. et al. (1998) Application of MRI for small intestinal diseases. J Magn Reson Imaging; 8:375-383; Madsen, S. M. et al. (1997) Magnetic resonance imaging of Crohn disease: early recognition of treatment response and relapse. Abdom Imaging; 22:164-166; Van Beers, B. et al. (1994) MRI of complicated anal fistulae: comparison with digital examination. J Comput Assist Tomogr; 18:87-90].

In addition, Shi W. et al. (1998) Q. J. Med; 91 295-301 report the localization of tumors with [¹¹¹In]DTPA-octreotide by scintigrapby and by MRI imaging.

The gastrointestinal (GI) tract also allows the introduction of imaging probes and contrasting agents relatively non-invasively, namely, per os or pro rectum. The use of imaging in combination with gastro-retentive imaging probes, which are to be retained in the stomach for a suitable period of time, have also been described.

Steingoetter et al.^{(1, 2)} describe the use of tablets with different floating characteristics and marked with iron oxide particles (such as super-paramagnetic Fe₃O₄ particles) in order to analyze intra-gastric tablet position and residence time in a subject. The super-paramagnetic Fe₃O₄ particles are images by the use of MRI techniques.

Shalaby et al.⁽³⁾ describes ultrasound and fluoroscopic imaging techniques in order to monitor the gastric retention of enzyme-digestible hydrogels in the canine stomach. The real-time fluoroscopic imaging was achieved by loading the gels with diatrizoate meglumine/sodium diatrizoate. The resulting hydrogels are described to have a low degree of deformation during peristalsis with long gastric retention times.

Devices that can be retained in the stomach for periods of 3 to 24 hours have been described in U.S. Pat. No. 6,685,962⁽⁴⁾.

### SUMMARY OF THE INVENTION

It is an object of the present invention to provide a gastro-retentive device for the delivery of a diagnostic utility, which may be retained in the GI tract, specifically in the stomach, for a period of time longer than the physiological gastric emptying time.

Thus, in accordance with a first aspect of the invention there is provided a gastro-retentive diagnostic assembly (GRDA) for use in determining a condition of the gastrointestinal tract (GI tract), preferably, of the stomach, comprising a folded single or multi-layered device comprising a diagnostic utility, the device prior to folding being essentially planar, and included in a delivery system for oral intake, the delivery system being adapted to release the device once in the stomach, whereupon release, said device unfolds into an unfolded shape that results in the retention of the device in the stomach.

The invention also provides a method of determining a condition of a subject's GI tract, preferably the stomach, the method comprises orally administering to a subject with a GRDA comprising a folded single or multi-layered device comprising a diagnostic utility, the device prior to folding being essentially planar, and included in a delivery system for oral intake, the delivery system being adapted to release the device once in the stomach, whereupon release, said device unfolds into an unfolded shape that results in the retention of the device in the stomach; and retrieving data indicative of a condition of the subject's GI tract.

The diagnostic method of the invention may be utilized for determining an abnormality in the GI tract as well as in other locations and organs along the GI tract, preferably in the stomach. Specifically, functional condition as well structural condition along the GI tract may be diagnosed. The method of the invention may also be utilized for monitoring a change in a condition in the GI tract, e.g. following or during a medical treatment. The medical treatment may include providing the subject with a medicament or performing a medical procedure so as to ameliorating the subject's state. For monitoring a change in a condition of the GI tract, the method of the invention may involve a several sequential administrations of the GRDA of the invention (intervals of hours, days, weeks or months), each followed by imaging of the GI tract.

The invention also provides the use of an essentially planar single or multi-layered device comprising a diagnostic utility for the preparation of a GRDA for oral intake, the GRDA comprising said device in a folded configuration within a delivery system, the single or multi-layered device being characterized in that when released from the delivery system, it unfolds into an unfolded shape that results in the retention of the device in the stomach.

The invention further provides a method for preparing a GRDA for use in diagnosing a condition of the GI tract, the method comprises: (i) providing an unfolded and essentially planar single or multi-layered device comprising a diagnostic utility; (ii) folding said device; and (iii) introducing the folded device into or combining it with a delivery system, such that when in the stomach it is released from the delivery system, whereupon release it unfolds into an unfolded shape that results in the retention of the device in the stomach.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A-1B are representative coronal MRI images of human subject without (FIG. 1A) or with (FIG. 1B) oral administration of the GRDA comprising a layer which is insoluble in gastric pH, carrying magnetite as the contrasting agent. The stomach is marked by a dotted line.

FIGS. 2A-2B are representative coronal MRI images of the same subject depicted in FIG. 1B, however, 10 hours (FIG. 2A) and 24 hours (FIG. 2B) after oral administration of the GRDA.

FIGS. 3A-3C are representative axial MRI images of the stomach of a human subject after a low-calorie meal without (FIG. 3A) or after taking (FIG. 3B) a soluble polymer-based GRDA carrying magnetite. FIG. 3C is an enlargement of an area in FIG. 3B (marked with an arrow).

FIGS. 4A-4B are respective, coronal and axial MRI images, of an insoluble polymer based GRDA in a subject's stomach under fast conditions.

FIGS. 5A-5B are respective, coronal and axial MRI images, of an insoluble polymer based GRDA given to the subject of FIG. 4A-4B, however, after being evacuated from the subject's stomach under fast conditions.

FIGS. 6A-6B are γ-scintigraphy images of a subject's stomach after receiving a ^{99m}Tc-labeled low calorie meal and after being administered with ¹¹¹In-labeled GRDA as observed simultaneously in the ^{99m}Tc channel (FIG. 6A) and the ¹¹¹In (FIG. 6B).

FIGS. 7A-7B are γ-scintigraphy images of a subject's stomach 1.5 hours after dosing. FIG. 7A shows 50% evacuation of the meal (^{99m}Tc channel) while the ¹¹¹In-labeled GRDA is still observed in the stomach (FIG. 7B).

FIGS. 8A-8B are γ-scintigraphy images of a subject's stomach 2.0 hours after dosing. FIG. 8A shows 90% evacuation of the meal (^{99m}Tc channel) while the ¹¹¹In-labeled GRDA is still observed in the stomach (FIG. 8B).

FIGS. 9A-9B are γ-scintigraphy images of a subject's stomach 5 hours (FIG. 9A) or 5.5 hours (FIG. 9B) after being administered with ¹¹¹In-labeled GRDA.

FIGS. 10A-10B are γ-scintigraphy images of a subject's stomach 3.75 hours (FIG. 10A) or 4 hours (FIG. 10B) after being administered with ¹¹¹In-labeled non-disintegrating tablet, used as control.

### DETAILED DESCRIPTION OF EXEMPLARY EMBODIMENTS

The present invention provides a GRDA comprising a folded single or multi-layered device comprising a diagnostic utility which is included or combined with a delivery system, for oral intake, such that once in the stomach, the device is released from the delivery system and unfolds to an unfolded shape yielding the retention of the device in the stomach.

As appreciated, while the invention is described in the following detailed description with reference to the diagnostic assembly and methods, it is to be understood that also encompassed within the present invention is the use of an unfolded single or multi-layered device comprising the diagnostic utility for the preparation of the GRDA of the invention, as well as methods of preparing the GRDA from the unfolded single or multi-layered device.

As used in the specification and claims, the forms “a”, “an” and “the” include singular as well as plural references unless the context clearly dictates otherwise. For example, the term “a diagnostic utility” includes one or more diagnostic agents and the term “a contrasting agent” includes one or more contrasting agents.

Further, as used herein, the term “comprising” is intended to mean that the layers of the device include the recited elements, but not excluding others. The term “consisting essentially of” is used to define layers that include the recited elements but exclude other elements that may have an essential significance on the diagnosing, e.g. imaging of die GI tract. For example, a device consisting essentially of soluble polymer based layer will not include or include only insignificant amounts (amounts that will have an insignificant effect on the release of the contrasting agent from the device) of polymers that prevent the dissolution of the matrix in the gastric fluid, such as enteric polymers. “Consisting of” shall thus mean excluding more than trace elements of other elements. Embodiments defined by each of these transition terms are within the scope of this invention.

Further, all numerical values, e.g. when referring the amounts or ranges of the elements constituting the device's layers, are approximations which are varied (+) or (−) by up to 20%, at times by up to 10% of from the stated values. It is to be understood, even if not always explicitly stated that all numerical designations are preceded by the term “about”.

The GRDA of the invention may be applicable for any of a variety of diagnostic techniques as known to those versed in the art. The selection of a suitable diagnostic technique will depend, inter alia, on the type of a diagnostic utility incorporated in the GRDA, the manner of administration and the condition to be diagnosed. In case the diagnostic technique is imaging, the GRDA may depend on the type of contrasting agent employed etc. Imaging techniques typically employed in medical diagnostics include, without being limited thereto, X-ray (computer tomography (CT) of CAT scan), ultrasound, γ-scintigraphy or MRI imaging.

According to one embodiment, the diagnostic technique is imaging. According to a particular, although not limiting, embodiment, the imaging technique is MRI. A variety of contrasting agents may be employed for the purpose of MRI. For example, agents which are paramagnetic, i.e. molecules which have unpaired electrons. In general, paramagnetic species may be simple substance (i.e. molecular oxygen), a stable radical (i.e. nitroxide radical) or a metal ion (i.e. many transition metal ions), the latter being most suitable for diagnostic imaging purposes.

Some paramagnetic metal ions include, without being limited thereto, Cr⁺³, Mn⁺², Fe⁺³, Cu⁺², Eu⁺³, Gd⁺³ and Dy⁺³. In order to reduce toxicity of these metal ions, they may be complexed with a carrier, such as the Gadolinium-DTPA complex.

Metalloporphyrines of iron(III) and manganese(III) are also used as contrasting agents. Porphyrins have been used in photodynamic therapy of tumors and their selective retention in tumor has led recently to their study as MRI contrasting media. Heme-containing proteins which act similarly to the porphyrins, are known as “natural” contrast agents.

A preferred type of paramagnetic contrasting agents for use in MRI includes the superparamagnetic iron oxide (SPIO) based colloids. These substances consist of nonstoichiometric microcrystalline magnetite cores which are coated with dextranes or siloxanes. There are a variety of SPIO reagents available on the market, known by their trademark as Feridex I.V™, Endorem™, Gastromark™, Lumirem™, Sinerem™.

Magnetite is a specific contrasting agent in accordance with the invention. Nonetheless, it is noted that the GRDA of the invention is non-agent specific and can serve as a platform to include a variety of contrasting agents as known in the art.

Other contrasting agent may include sucrose polyesters, nanometric superparamgnetic iron oxide (mixtures of Fe₂O₃ and Fe₃O₄) imbedded in monodispersed polymers or a protein or an inert silicon polymer as well as others known in the art.

It is noted that mineral oils, oil emulsions as well as combinations of oil emulsions and substances described above may also be employed as a contrasting agent. For example, an emulsion containing corn oil and ferric ammonium citrate, and an emulsion containing baby formula with ferrous sulfate were described. These are palatable mixtures that distribute uniformly in the bowel and thus may be used for diagnosing bowl conditions.

As used herein the term “diagnostic utility” denotes a single or combination of elements which provide data or information representing a condition within the GI tract, or from which information regarding the GI tract may be deduced.

According to one embodiment, the diagnostic utility comprises, at minimum, a contrasting agent to be used in imaging of the GI tract by any imaging technique available in the art.

Nonetheless, the diagnostic utility may comprise in addition, or alternatively, other diagnostic elements which enable the detection of a condition of the GI tract. For example, without being limited thereto, the diagnostic utility may comprise a pH sensor for sensing the pH or pH changes within the GI tract, an acoustic sensor (e.g. a piezo-electric sensor), for sensing acoustic sounds emanated from the GI tract, a temperature sensor, a pressure-sensing device, a blood-detecting device etc. In addition, the diagnostic utility may comprise additional components such as a telemetry device (e.g. a transmitter powered by a low power energy source) for transmitting an output indicative of the GI tract's condition to a remote external receiver or recorder, a memory unit for recording and storing the data outputted from telemetry device.

The diagnostic utility may be used as is, or they may be associated with a carrier or delivery vector. The carrier or vector is designed to allow interaction of the diagnostic utility with the GI tract, e.g. with the stomach's lumen. The carrier or vector may be releasable or non-releasable from the single or multi-layered device and it may be a biological substance such as a peptide, a protein (e.g. antibody), a polysaccharide, a liposome or a cell, preferably having some degree of affinity to the gastric lumen (e.g. to receptors or antigens presented on the lumen), so as to direct the diagnostic utility to the lumen. Further, it is to be noted that the diagnostic utility may be provided in a form of a particle, such as an aggregate or a colloidal particle or be incorporated in a particle.

The term “interaction” or “interact” denotes the pharmacokinetic and/or pharmacodynamic behavior of the diagnostic utility in the GI tract. It includes, for example: metabolism or breakdown with the GI tract; absorption by cells (including bacterial cells), tissue or disease-causing agents in the GI tract; distribution or diffusion within the GI tract; binding to substance or molecules within the GI tract, e.g. such secreted by certain cells or tissue; etc.

The delivery system incorporating therein the single or multi-layered device when the latter is in a folded configuration may be any pharmaceutically acceptable orally delivered container, as known in the art of pharmaceutical delivery vehicles. The container may be, without being limited thereto, a capsule (soft or solid) containing the folded device, an elongated tube, a ring or a thread (one or more) surrounding the folded device, a polymeric coating (e.g. a polymeric thread wrapping the device in a manner resembling a cocoon), a polymer or gel matrix embedding the folded device and the like. The single or multi layered device may be released from the delivery system as a result of the dissolution or breakdown of the delivery system when wetted by gastric fluids. A preferred container in accordance with the invention is a hard gelatin capsule.

It is noted that in accordance with the invention, the GRDA is applicable for in vivo as well as in vitro applications. When administered to a human subject (for in vivo applications) the single or multi-layered device is released from the delivery system as a result of dissolution or breakdown of the delivery system when wetted by the gastric fluids. However, gastric fluids may be simulated in vitro by the use of suitable aqueous buffers.

As used herein, the term “folded” denotes any manner known in the art to reduce an effective projection surface:volume ratio of a generally planar layer, and includes, without being limited thereto, one or more of folding about fold lines, bending, twisting, wrapping, winding, rolling, crimping and the like.

In some preferred embodiments, the single or multi-layered device is folded parallel to the width of the unfolded device and designed to have folds which are symmetric mirror images about a first axis. This manner of folding provides an accordion-like configuration for the device.

According to another embodiment, the folded form of the device has folds of increasingly smaller amplitudes upon extending away from the first axis so as to form a partially rounded cross section and to allow the folded form to easily be inserted into a container.

According to yet another embodiment, the folded form of the device has folds of increasingly larger amplitudes upon extending away from one end of the first axis to its other end, so as to form a fan-like configuration.

In the context of the invention, the term “unfolded” denotes an essentially and generally planar configuration of the device. The term “essentially planar” or “generally planar” denotes a fully planar as well as wiggly or wavy shape of the device. Unfolding denotes any form of expansion of the device, which may result form unwinding, unrolling, inflating, swelling, and the like. Following expansion in the stomach, the unfolded and essentially planar device maintains its firmness due to its unique characteristics, as exemplified below.

The term “gastro-retentive” or “gastro-retentivity” as used herein denotes the maintenance or withholding of a contrasting agent in the GI tract (either after being released from or still in association with the single or multi-layer device), for a time period longer than the time it would have been retained in the stomach when delivered in a free form or within a gastrointestinal delivery vehicle which is not considered gastro-retentive. Gastro-retentivity may be characterized by retention in the stomach for a period that is longer than the normal emptying time from the stomach, i.e. longer than about 2 hours, particularly longer than about ˜3 hours and usually more than about 4, 6, 8 or 10 hours. Gastroretentivity typically means retention in the stomach from about 3, 4, 6, 8 or at times 10 hours up to about 18 hours. It is however noted that in accordance with the invention, retention of the GRDA is not observed after more than 48 hours after administration, and preferably not after 24 hours.

The GRDA is administered to the stomach, preferably by swallowing. Once it is wetted in the gastric lumen (by the gastric fluids), the single or multi-layered device comprising the diagnostic utility is released from the delivery system and unfolds to a configuration which permits the retention of the device in the stomach for a time sufficient for retrieving data indicative of a condition of the stomach. The time is preferably longer than the physiological gastric emptying (when ingested following fast or a low calorie meal).

According to one embodiment, the diagnostic utility remains fixed in and is not released from the device during the determination period. According to another embodiment, the diagnostic utility is released from the device once in the stomach. The release of the diagnostic utility from the device may have a controlled release profile.

According to another embodiment, the contrasting agent is released from the device at a relatively slow rate so as to permit imaging of the device and the GI lumen (e.g. when the diagnostic utility is designed to associate with the GI luman, e.g. by attaching thereto a ligand having affinity to a specific receptor presented in the lumen) throughout a substantial portion of time of the device's retention in the stomach as indicated above. A substantial portion of time includes at least 2 hours, particularly longer than about 3 hours and usually more than about 4, 6, 8, 10 or 18 hours.

The GRDA may have a variety of applications in medical diagnostics. It is preferably employed for the imaging of the GI tract, for the purpose of diagnosing or monitoring a pathological condition of the GI tract, for monitoring the (normal or abnormal) function of the GI tract, for monitoring treatment of the GI tract and/or for evaluating GI transit time in a subject in need.

In accordance with the invention, a “condition of the GI tract” used interchangeably with the term “pathological condition” of the GI tract denotes any condition of the GI tract, preferably the stomach, which is associated with an abnormality of the GI tract. This includes a disorder or disease where the primary abnormality of the GI tract is an altered physiological function (the way the body works) such as in the case of irritable bowel syndrome (IBS) and dyspepsia (which are the most common functional GI disorders), as well as structural disorders (having an identifiable structural or biochemical cause, such as in the case of GI polyps, cancer, ulcer etc.).

The following is a non-limiting list of pathological conditions that may be diagnosed by the use of the GRDA of the invention:
Stomach-origin anomalies, such as, without being limited thereto, gastroparesis, gastritis, gastroenteritis—viral or bacterial, gastric ulcer (e.g. peptic ulcer disease), gastric cancer;
Intestinal-origin anomalies, such as, without being limited thereto, irritable bowel syndrome, GI bleeding, GI portal hypertension (viewed by the appearance of varices) colitis, diverticulosis, colon polyps, GI cancer, carcinoid, inflammatory bowel disease (IBD), GI obstructions, metabolic diseases associated with excess or deficient secretion of gut hormones such as gastrin, motilin, cholicytokinin (CCK), somatostatin, secretin, vasoactive intestinal peptide (VIP), galanin, geralin, and enzymes such as amylase, lipase, pepsins, chymotrypsin and trypsin.

While commonly used contrasting agents are mainly administrated intravenously to image the GI tract, the GRDA of the invention is orally administered to the subject in need. The GRDA may be provided to the subject after fasting or after eating. It is preferable however that the GRDA is provided after a low calorie meal.

The invention also provides a method of determining a condition of a subject's GI tract, the method comprises providing said subject with the GRDA of the invention and retrieving data indicative of the condition of subject's GI tract by the use of a suitable diagnostic technique. As indicated above, the GRDA may be administered to the subject following fast or a meal (preferably a low calorie meal). Following administration of the GRDA the technician or physician can perform a suitable detection method, e.g. imaging, in order to obtain data indicative of the condition. The term “data indicative of a condition” should be understood in correlation with the type of diagnostic utility employed. For example, when the diagnostic utility is provided with a contrasting agent, the data retrieved will comprise one or more images of the GI tract, preferably the stomach, as further detailed below. When the diagnostic utility comprises an acoustic sensor, the data retrieved may be a signal or stream of signals indicative of movements at the area of the sensor. Further, as an example, when the diagnostic utility comprises a pH or temperature sensor, the data respectively corresponds to the pH or temperature within the GI tract. It is noted that the data may be a single parameter, e.g. a single pH or temperature value, a single image of the GI tract, or a series of parameters measured at different time points during the detection procedure.

When the diagnostic utility comprises a contrasting agent, the physician or technician may also trace its trail through the GI tract with the assistance of imaging systems and techniques such as those known in the art. To this end, one or more individual images may be captured at different time points throughout the procedure as well as capturing a sequence of images, e.g. to obtain video data stream of the GI tract. The images obtained by the imaging system may then be analyzed by a radiologist or gastroenterologist as known in the art.

The invention also provides the use of an unfolded as well as folded single or multi-layered device comprising a diagnostic utility for the preparation of a GRDA and methods for preparing the GRDA, both as described herein above and below. In the broadest sense, the preparative method comprises providing an unfolded and essentially planar single or multi-layered device comprising a diagnostic utility; folding the device; and introducing the folded device within or combining it with a delivery system, such that when in the stomach it is released from the delivery system, whereupon release, it unfolds into an unfolded shape that results in the retention of the device in the stomach.

As described above, the GRDA comprises a folded single or multi-layered device. According to one embodiment, the GRDA comprises a single layered device folded within the delivery system. To this end, the diagnostic utility may be adsorbed to a surface (one or both surfaces) of the layer, or embedded or entrapped within the layer (e.g. the layer being impregnated with a diagnostic utility) or absorbed into a carrier, such as a string or a thread that is attached to the GRDA (e.g. as a sting circling the device).

According to another embodiment, the GRDA comprises two or more layers sandwiching the diagnostic utility therebetween. The diagnostic utility, in accordance with this embodiment, may also be adsorbed to one more surfaces of the multi-layered device, it may be sandwiched within all or only part of the devices' layers, it may be absorbed into a carrier, such as a string or a thread that is attached to the GRDA or a combination of layers having a diagnostic utility embedded in the layer, with layers having the diagnostic utility adsorbed to or sandwiched between the layers or absorbed into a string or a thread that is attached to the GRDA.

According to one embodiment, the device's layer(s) comprise a matrix carrying the diagnostic utility. The diagnostic utility may be embedded or entrapped within the matrix or adsorbed or affixed to a surface of the matrix. The matrix may comprise one or more polymers, including, without being limited thereto, polymers soluble in gastric fluids, polymers insoluble in gastric fluids, as well as a combination of at least one such soluble polymer and at least one such insoluble polymer.

As used herein, the term “insoluble polymer” denotes a polymer that when immersed in gastric fluids at 37° C. it does not lose more than 10% of its dry weight into the medium by dissolution. Consequently, films made of one or more insoluble polymers will preserve their shape in gastric fluid for at least 2 hours.

The term “soluble polymer” as used herein denotes a polymer that forms a hydrogel or dissolved in gastric fluids at 37° C. In this connection, the term “hydrogel forming polymer” denotes a polymer or a mixture of polymers that once in gastric fluid, absorb an amount of gastric fluid which results in the formation of a gel phase within the GRDA.

According to one embodiment, the polymer soluble in gastric content comprises one or more polymers selected from a hydrogel-forming polymer, a non-hydrogel polymer, or any combination thereof. Non-limiting examples of hydrogel-forming polymer comprise proteins, polysaccharides, including gums (e.g. carrageenans, ceratonia, acacia, tragacanth, guar gum and xanthan gum), gelatine, chitosan, polydextrose, cellulose derivatives, such as high molecular weight grades of hydroxypropyl cellulose, hypromelose, hydroxyethyl methyl cellulose, methyl cellulose, polyethylene oxides, polyvinyl alcohol and derivatives of any one of the above which are soluble in gastric fluid as well as any combination of two or more thereof, the combination also being soluble in gastric fluid.

Non-limiting examples of non hydrogel-forming polymer comprise povidones (PVP), methacrylic acid copolymer with dimethyl amino ethyl methacrylate (Eudragit E™), low molecular weight grades of hydroxypropyl cellulose, propylene glycol alginate, polyethylene glycols, poloxamers and soluble derivatives of any one of the above as well as any combination of two or more thereof.

According to another embodiment, the matrix comprises a polymer that is insoluble in gastric content. The insoluble polymer comprises one or more polymers selected from an enteric polymer, a non-enteric polymer, or any combination thereof. An enteric polymer is preferably such that it is substantially insoluble at a pH of less than 5.5. Non-limiting examples of enteric polymers applicable with respect to the invention include, shellac, cellacefate, hypromelose phthalate, hydroxypropyl methylcellulose acetate succinate, zein, polyvinyl acetate phthalate, aliginic acid and it's salts, carboxymethyl cellulose and it's salts, methylmethacrylate-methacrylic acid copolymers, including ethyl acrylate copolymers, or substantially insoluble derivatives of any one of the above as well as any appropriate combination of two or more of the above. Non-limiting examples of non-enteric polymers applicable with respect to the invention include poly(lactide), poly(glycolide), poly(lactide-co-glycolide), ethylcellulose; cellulose acetate; a copolymer of acrylic acid and methacrylic acid esters, having of from about 5% to about 10% functional quaternary ammonium groups; a polyethylene; a polyamide; a polyester; a polyurethane, polyvinylchloride; polyvinyl acetate; and a combination of any two or more thereof.

The desired configuration of the single or multi-layered device, once unfolded, may be achieved by the incorporation of an enforcing polymeric composition having a mechanical strength enabling the preservation of the unfolded configuration of the device, i.e. after ingestion. The enforcing polymeric composition may be provided over the polymeric matrix or may be integrally formed with the matrix.

According to one embodiment, the enforcing polymeric composition is in the form of one or more continuous or non-continuous polymer strips. For example, the strips may define a continuous or non-continuous frame at said device's periphery. The continuous or non-continuous frame may be either affixed or attached to the matrix or integrally formed with the matrix. In the context of the invention, it is to be understood that the matrix and the enforcing polymeric composition together form the devices' layer(s).

To provide the desired enforcement in its unfolded state, it is preferable that the polymeric composition comprises an enteric or non-enteric polymer, insoluble in gastric content or a combination of enteric and non-enteric insoluble polymers. Pharmaceutically acceptable enteric and non-enteric insoluble polymers are known and readily available to those versed in the art. An enteric polymer is preferably such that it is substantially insoluble at a pH of less than 5.5. Non-limiting examples of enteric polymers applicable with respect to the invention include, shellac, cellacefate, hypromelose phthalate, hydroxypropyl methylcellulose acetate succinate, zein, polyvinyl acetate phthalate (although being soluble at a pH above 4.6), aliginic acid and it's salts, carboxymethyl cellulose and it's salts, methylmethacrylate-methacrylic acid copolymers, including ethyl acrylate copolymers, or substantially insoluble derivatives of any one of the above as well as any appropriate combination of two or more of the above.

Non-limiting examples of non-enteric insoluble polymers applicable with respect to the invention include poly(lactide), poly(glycolide), poly(lactide-co-glycolide), ethylcellulose; cellulose acetate; a copolymer of acrylic acid and methacrylic acid esters, having of from about 5% to about 10% functional quaternary ammonium groups; a polyethylene; a polyamide; a polyester; a polyurethane, polyvinylchloride; polyvinyl acetate; and a combination of any two or more thereof.

In addition to the above mentioned polymeric composition, the enforcement may be achieved by combining in the polymeric composition an insoluble polymer with a further polymer, soluble in gastric content. The soluble polymer may be entrapped in the insoluble polymer or it may be modified, for example by cross-linked with the insoluble polymer, in such way that it does not exude from the polymer composition, unless disintegrating of the whole enforcing polymeric composition. Non-limiting list of soluble polymers which may be combined with the insoluble polymer, forming together the enforcing polymeric composition, comprises proteins, polysaccharides, including gums (e.g. carrageenans, ceratonia, acacia, tragacanth, guar gum and xanthan gum), gelatine, chitosan, polydextrose, cellulose derivatives, such as hydroxypropyl cellulose, hypromelose, hydroxyethyl methyl cellulose, methyl cellulose; polyethylene oxides, polyvinyl alcohols, povidones (PVP), methacrylic acid copolymer with dimethyl amino ethyl methacrylate (Eudragit E™), propylene glycol alginate, polyethylene glycols, poloxamers, and soluble derivatives of any one of the above as well as any combination of two or more thereof.

The device of the invention may also comprise external shielding sheets. The external sheets may comprise one or more polymers selected from the group consisting, without being limited thereto, polymers soluble in gastric content, polymers insoluble in gastric content, and a combination of any two or more thereof.

According to another embodiment, the external sheet is comprised of a mixture of a soluble polymer and an enteric polymer. According to yet another embodiment, the external sheet comprises a cross-linked soluble polymer, e.g. an enzymatically hydrolyzed cross-linked gelatin and a derivative thereof.

Another example of external sheet composition can be polyvinyl alcohol film, cross-linked with glutaraldehyde. Alternatively, said polyvinyl alcohol film could be subjected to one or more freeze-thaw cycles to induce crystallization.

Another example of external sheet composition can be polyethylene oxide film, cross-linked by gamma irradiation.

In yet another example the external sheet may comprise polydimethyl siloxane and its derivatives.

Those versed in the art will know how to select the specific polymers forming the device of the invention, while considering the following basic criteria:

It is essential that the GRDA comprises at least one insoluble polymer. The insoluble polymer may be in the matrix, the enforcing polymeric composition or, if present, in the shielding, sheet. Preferably, the insoluble polymer forms part of the enforcing polymeric composition.

When release of the diagnostic utility is desired, it is preferably embedded in a soluble polymer, and motatis mutandis, an insoluble polymer will be preferable when gastro-retention of the diagnostic utility in the device is desired. Evidently, a combination of soluble and insoluble polymers may be used and the ratios therebetween will depend on the characteristics required for the inner matrix.

As disclosed herein, the enforcing polymeric composition preferably provides the mechanical properties of the device. Thus, the enforcing layer may be characterized by a flexural strength and both between 25 and 100 kgf/mm².after immersion in simulated gastric fluid. Thus, as also disclosed hereinabove, it is preferable that the enforcing layer comprises at least one insoluble polymer. However, it should be noted that soluble polymers may be used to form the enforcing layer provided that are interacted so that the film becomes insoluble through either chemical or physical cross linking, or by coating them with and insoluble polymer.

It is also essential that the shielding sheet comprises polymers that are permeable to the gastric fluid and to allow release of substance from the device, if such release is desired. The shield should, however, be impermeable to soluble polymers of the matrix since the shield should facilitate the existence of a separate compartment inside the device that has a different composition from that of the GI tract for the duration of its activity.

It is also preferable that at least one layer in the device comprises a swellable polymer (hydrogel) to facilitate the unfolding of the device.

To further facilitate the unfolding of the diagnostic device once wetted by gastric fluids, e.g. fluids in the stomach, an anti-adhering material may be applied to at least a portion of the outer surfaces of the device. Alternatively, the device may be covered with external ‘shielding’ sheets and the anti-adhering material may be provided over at least a portion of the external sheets.

The anti-adhering material may be such material as known to those versed in the art. Examples include, without being limited thereto, pharmaceutically acceptable celluloses, cellulose derivatives, silicates, glyceryl esters of fatty acids and others, or water repelling agents, i.e. simethicone, dimeticone, cyclomethicone and others. A preferred anti-adhering material comprises microcrystalline cellulose.

The GRDA of the invention may also comprise a plasticizer. Examples of plasticizers include, without being limited thereto, citrate esters, phthalate esters, dibutyl sebacate, diacetylated monoglycerides, glycerin, glycerin derivatives (such as triacetin), polyethylene glycols, propylene glycol, sorbitol, or a combination of such plasticizers.

Further, the GRDA of the invention may comprise a filler. The filler may be starch, glucose, lactose, an inorganic salt, a carbonate, a bicarbonate, a sulfate, a nitrate, a silicate, an alkali metal phosphate, an oxide, or a combination thereof.

In addition to the mentioned composition, the device may comprise lubricants, and other pharmaceutically acceptable processing adjutants, as known in the art.

The invention will now be exemplified in the following description of experiments that were carried out to exhibit the utility of the GRDA of the invention in imaging. It is to be understood that these examples are intended to be in the nature of illustration rather than of limitation. Obviously, many modifications and variations of the GRDA exemplified are possible in light of the above teaching. It is therefore, to be understood that within the scope of the appended claims, the invention may be practiced otherwise, in a myriad of possible ways, than as specifically described hereinbelow.

### Some Exemplary Embodiments

### Preparation of a Gastro-Retentive Diagnostic Assembly (GRDA) Comprising a Contrasting Agent

The GRDA exemplified herein is composed of three layers, the central layer contains a polymer-contrasting agent matrix (the matrix detailed below) and continuous strip (in a frame shape) of enforcing polymeric composition, and it was covered on both sides with hydrolyzed gelatin based polymeric layers, the properties of which are controlled by the degree of cross-linking with glutaraldehyde. The GRDA was of oval shape, 45 mm long by 24 mm wide (at its widest point) before folding into an E00 hard gelatin capsule. For the MRI studies, the matrix was mixed with magnetite. The matrix comprised either a polymer that is soluble in the gastric fluid or a polymer that is essentially non-soluble in the gastric fluid, as specifically exemplified below.

In all examples, strips of enforcing polymeric composition were prepared by casting a solution consisting of methylmethacrylate-methacrylic acid copolymer (50%, Eudragit L100, Degussa), ethylcellulose N100 (20%, Hercules) and triacetin (30%, Merck) in ethanol.

The shielding sheet was prepared by casting a solution consisting of enzymatically hydrolyzed gelatin (24%, average molecular weight 10,000-12,000, Byco E, Croda), methylmethacrylate-methacrylic acid copolymer (30%, Eudragit S, Degussa) and glycerin (30%) in a mixture of 50% ethanol and 50% NaOH—K2HPO4 buffer. Glutaraldehyde (2%, Merck), diluted in the same solvent, was added whilst mixing before casting for cross linking and evaporation.

The resulting films (matrix, enforcing strops and shielding sheets) were cut to size with an appropriate template or dice, and assembled together to form the GRDA, applying (by brush or spray) ethanol as an adhesive. The laminated, essentially flat GRDA, was sprayed with ethanol and powdered with microcrystalline cellulose (Avicel, FMC BioPolymers) on both external faces. The powdered laminate was then folded into a hard gelatin capsule (E00, Capsugel).

For γ-scintigraphy a placebo matrix made of hydroxypropyl cellulose was used. After assembling and folding of the GRDA and a cotton thread was sawn into the GRDA so as to wrap several times around the strip of the enforcing polymeric composition, leaving about 2 cm long pending. This pending thread was used by the radioactive labeling laboratory to label the GRDA. The thread was dipped into a 0.05M solution of ¹¹¹InCl₃ to allow soaking of the cotton thread by capillary force. The thread was air dried, and was then dip coated with a solution consisting of 50 ml acetone, 50 ml isopropyl alcohol, 0.25 g triacetin and 4.75 g ethylcellulose to fix the indium salt in the thread.

Once prepared, the GRDA was folded and enclosed in a hard gelatin capsule (CAPSUGEL).

### Preparation of Non-Soluble Matrix

Non-soluble matrices were prepared from polymethyl methacrylate-polymethyl methacrylic acid copolymer. The contrasting agent of choice was magnetite which was loaded into the polymeric matrix by forming a dispersion of the agent in a dissolved polymer solution. The dispersion was then cast onto trays and dried in an oven.

Two types of essentially non-soluble matrices were prepared:

(i) Polymethyl methacrylate-polymethyl methacrylic acid copolymer (1;1) (Eudragit® L, Roehm, 57 g) dissolved in Ethanol USP up to 700 ml and then mixed with PEG 20,000 (Fluka, 38 g) and with magnetite (Black iron oxide, Aldrich, 5 g or BASF) until a homogeneous dispersion was obtained, cast into trays and dried.

(ii) Polymethyl methacrylate-polymethyl methacrylic acid copolymer (1:1) (Eudragit® L, Roehm, 57 g) dissolved in Ethanol USP up to 700 ml and then mixed with magnetite (Aldrich, 5 g), ^{20,000}PEG 20,000 (Fluka, 38 g) and with Triacetin [Fluka or Merck, 29.55 g] until a homogeneous dispersion was obtained, cast into trays and dried.

### Preparation of a Soluble Polymer Matrix:

A soluble polymer matrix was prepared from hydroxypropyl cellulose film.

The contrasting agent of choice was magnetite which was loaded onto the film by dissolving the hydroxypropyl cellulose (Klucel® EF, Hercules, 95 g) in water (Distilled water, up to 1 liter) and adding magnetite (Black iron oxide, 5 g; Aldrich), to the dissolved hydroxypropyl cellulose while stirring and until a homogeneous dispersion was obtained.

The dispersion was then cast onto trays and dried in an oven.

In a different procedure, the soluble polymer film was also prepared by dissolving hydroxypropyl cellulose (Klucel® EF, 98 g) in ethanol (Ethanol USP up to 1 liter) and adding magnetite (black iron oxide particles, 2 g) to the dissolved hydroxypropyl cellulose while stirring, until a homogeneous dispersion was obtained.

### Example 1

### Implementation of the GRDAs in MRI in Human Subjects

### Study Protocol:

In a Helsinki approved study conducted in two Israeli hospitals, informed volunteers (of both genders, total n=30) were requested to fast the night before the experiment (8 to 12 hours fast). The GRDA was taken orally with a glass of water (200 ml) immediately after a standardized low calorie meal (282 Kcal).

A series of MRI images was performed before and 30 min, 1.5, 3, 4.5, 6, 7.5, 9, 10.5, 12 and 24 h after GRDA administration. Five hours post-dosing an additional meal (˜900 kcal) was provided.

Retention of the GRDA capsule in the stomach was assessed by MRI at various time-points. Imaging of the volunteers was performed in supine position using the General Electric 0.5 T MRI machine (Sigma SP/I). Images were taken in axial and coronal planes.

The following MRI parameters were used:

1. localizer (0:56 min)

2. Fast Spoiled Gradient Echo (FSPGR), Axial: (1:41 min), Field of View (FOV): 40×40 cm; matrix size: 256×160; slice thickness: 6 mm×1 mm spacing; TE/TR=minimum/125; flip angle=80; BW=31.25; 5 NEX.

3. FSPGR, Coronal: (1:41 min); FOV: 46×46 cm; matrix size: 256×160; slice thickness: 6 mm×1 mm spacing; TE/TR=minimum/125; flip angle=80; BW=31.25; 5 NEX.

### Results

### MRI Imaging of GRDA

The MRI technique was shown to be a suitable method to determine the location of the magnetite-labeled GRDA in the GI tract and to assess the degree of retentivity of the GRDA in the stomach of human volunteers. The use of MRI provided an opportunity to closely follow the administered GRDA without any health hazard to the volunteer.

Two types of GRDAs were employed: a non-soluble polymer-based GRDA and a soluble polymer-based GRDA. Both devices where enclosed in a hard gelatin capsule and orally administered to the subjects concomitant with drinking a glass of water.

### Non-Soluble Polymer-Based GRDA

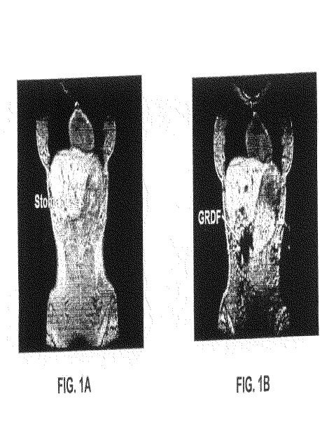

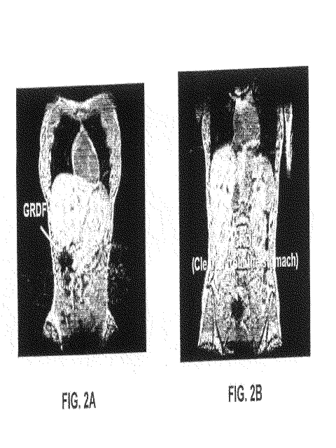

The positioning of the non-soluble polymer based GRDA is visualized in FIGS. 1A-1B. Specifically, FIG. 1A is a coronal MRI image a human subject 10 minutes after bread-fast (282 kcal), showing the contours of the stomach (dotted line), and the lungs and the heart of the subject above the stomach. FIG. 1B is a corresponding image of a subject's stomach dosed with the Non-soluble polymer based GRDA 10 minutes after the same breakfast (as in FIG. 1A). The GRDA is observed as a dark area in the lower part of the stomach. It is noted that some air is observed in the upper part of the stomach; however, it is possible to discern the GRDA from the air. Thus, FIGS. 1A-1B clearly show that the GRDA reaches the stomach upon oral delivery and may be visually observed by imaging techniques.

The retention and location in the stomach of the non-soluble polymer based GRDA 10 and 24 hours after oral intake were also images. FIG. 2A and FIG. 2B are MRI images of the subject's stomach and the GRDA at the indicated time points, respectively. Specifically, it is shown that after 10 hours the GRDA still resides in the stomach. However, after 24 hours, the GRDA is cleared from the stomach.

In a further study that was conducted according to the protocol described above, 6 healthy human subjects of both genders (ages 21 to 42) were used. Table 1 summarizes the times that the GRDA remain in each subject's stomach.

TABLE 1
GRDA imaging

| | MRI | | | | | | |
|---|---|---|---|---|---|---|---|
| Code | 3 h | 4.5 h | 6 h | 7.5 h | 8.5 h | 9.5 h | 24 h |
| 1 | + | + | + | + | + | + | + |
| 2 | + | + | − | NP | NP | NP | NP |
| 4 | + | + | − | NP | NP | NP | NP |
| 5 | + | + | + | + | + | + | − |
| 6 | + | + | + | + | + | + | − |
| 7 | + | + | + | + | + | + | − |
| Total | 6 | 6 | 4 | 4 | 4 | 4 | 1 |
| retained | | | | | | | |

+ GRDF in the stomach;
− GRDF is not in the stomach;
NP—not performed

The results presented in Table 1 clearly show that the GRDA is retained in the stomach for at least 10 hours and is removed from the stomach after 24 hours.

In yet a further study, 8 human subjects were given GRDA as described above, and imaged by MRI 4.5, 6, 8, 9.75 and 24 hours post dosing. The retention times in the stomach are summarized in the Table 2.

TABLE 2
GRDA imaging

| | MRI | | | | |
|---|---|---|---|---|---|
| Code | 4.25 h | 6 h | 8 h | 9.75 h | 24 h |
| 1 | + | + | + | + | − |
| 2 | + | + | + | + | − |
| 4 | + | − | − | − | − |
| 5 | + | + | + | + | − |
| 6 | − | − | − | − | − |
| 7 | + | + | + | + | − |
| 8 | + | + | + | + | − |
| 9 | + | + | + | + | − |
| Total | 7 | 6 | 6 | 6 | 0 |
| retained | | | | | |

+ GRDF in the stomach;
− GRDF is not in the stomach;
NP—not performed

The results presented in Table 2 confirm that the GRDA is retained in the stomach for at least 10 hours and is removed therefrom after 24 hours.

The MRI imaging technique with the GRDA was also evaluated under fasting conditions according to the FDA protocol for the evaluation of drugs, in comparison with dosing after a light breakfast as described above.

Specifically, 8 healthy human subjects of both genders were included (age range 23-44). All subjects were dosed with the GRDA after 10 hours of fasting. Four subjects were not given breakfast and were given a lunch of ˜900 kcal at 4 hours post dosing. The other 4 subjects followed the fed protocol described above. One week later the groups were reversed.

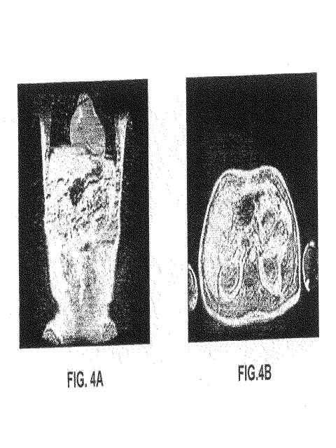

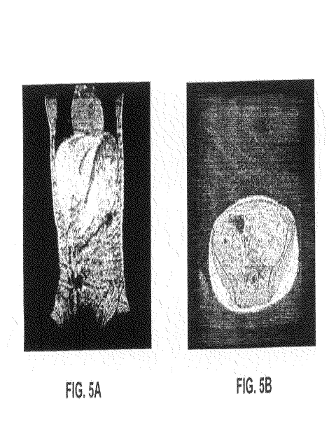

FIGS. 4A-4B and FIGS. 5A-5B demonstrate the ability to follow the GRDA under fasting conditions: FIG. 4A is coronal MRI image showing the GRDA in a fasted stomach, FIG. 4B is the respective axial image; FIG. 5A in a coronal MRI image showing the GRDA after it had left the stomach; FIG. 5B is the corresponding axial image.

Individual results of fast and fed protocols are presented in the Table 3.

TABLE 3
Fast vs. fed conditions

| | | Fast | | | | | | |
|---|---|---|---|---|---|---|---|---|
| | | 2 h | 3 h | 5 h | 7 h | 10 h | 13 h | 24 h |
| | | Fed | | | | | | |
| Code | | | 3 h | 5 h | 7 h | 10 h | 13 h | 24 h |
| 001 | Fast | − | − | NP | NP | NP | NP | NP |
| | Fed | NP | + | − | − | NP | NP | NP |
| 002 | Fast | + | + | + | + | + | + | +* |
| | Fed | NP | + | + | + | + | + | +* |
| 003 | Fast | + | − | − | NP | NP | NP | NP |
| | Fed | NP | − | − | NP | NP | NP | NP |
| 004 | Fast | + | + | + | + | + | + | − |
| | Fed | NP | + | + | + | + | + | − |
| 005 | Fast | − | − | NP | NP | NP | NP | NP |
| | Fed | NP | + | + | − | − | NP | NP |
| 007 | Fast | + | + | − | − | NP | NP | NP |
| | Fed | NP | + | + | + | + | + | − |
| 008 | Fast | − | − | NP | NP | NP | NP | NP |
| | Fed | NP | + | − | − | NP | NP | NP |
| 009 | Fast | + | − | − | NP | NP | NP | NP |
| | Fed | NP | + | + | + | + | + | − |
| Total | Fast | 5 | 3 | 2 | 2 | 2 | 2 | 1 |
| retained | Fed | NP | 7 | 5 | 4 | 4 | 4 | 1 |

+ GRDF in the stomach;
− GRDF is not in the stomach;
NP—not performed.
*evacuation of the GRDF from the stomach was confirmed at ~48 hours

It is noted that under fasting conditions stomach content and size, as well as motility patterns are different from those of the fed mode. Thus, it was important to demonstrate that imaging of the GRDA may be achieved under both conditions. It is of particular importance for diagnosing pathological conditions of the GI, which necessitates fast conditions. The results presented in Table 3 above thus show that the GRDA of the invention may be applicable also with fasted subjects.

### Soluble Polymer-Based GRDA

The presence of GRDA based on a soluble polymer (hydroxypropyl cellulose) was also examined. Specifically, a healthy human subject was given, 15 minutes after a low calorie meal (˜280 kcal,), a magnetite labeled soluble polymer based GRDA as described above (study protocol). Approximately 3 hours after dosing, the subject was imaged by MRI.

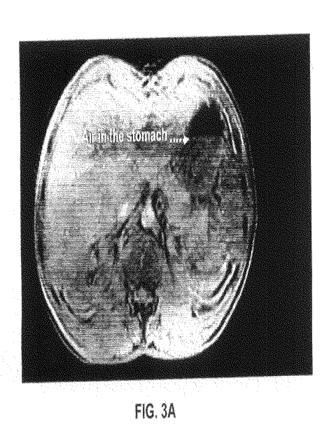

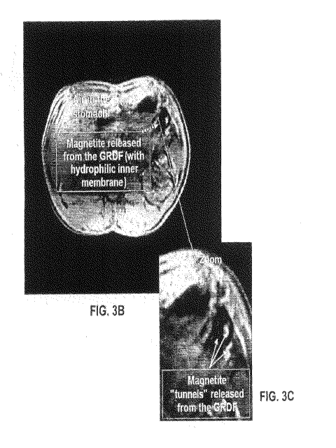

FIGS. 3A-3B are representative axial MRI images of the stomach of the subject after a low-calorie meal without the soluble polymer based GRDA (FIG. 3A) or after taking the GRDA (FIG. 3B). FIG. 3C is an enlargement of an area in FIG. 3B (marked with an arrow).

It is further noted from FIGS. 3A-3C that air in the stomach is visualized as a black area. Nonetheless, it is possible to distinguish in the stomach the presence of air from the presence of the magnetite. This is exemplified in the current extreme case where the GRDA is composed of an essentially fast dissolving polymer (i.e. highly soluble polymer) and thus allowed leakage of the magnetite from the GRDA, as seen in FIG. 3B and FIG. 3C

### Example 2

### Implementation of the GRDAs in γ-Scintigraphy in Human Subjects

The purpose of this study was to investigate the movement of the soluble-polymer based GRDA and its retention in the stomach (compared to a control tablet), and to verify that the GRDA does not interfere with normal food evacuation. Radioactively-labeled GRDA was followed in the stomach and through the GI tract of healthy subject, using a similarly labeled non-disintegrating tablet as a control. Each volunteer was dosed once with the GRDA and once with the control tablet (a week apart), in a randomized order. Prior to dosing the subjects were given a light breakfast labeled with a different radioactive element. Thus, it was possible to follow evacuation of the food simultaneously with the movement of the radio-labeled GRDA or the control tablet.

### Study Protocol:

In a study approved by the Local Ethics Committee and conducted to Good Clinical Practice in a hospital in Scotland, Informed subjects (males, total n=8) were requested to fast the night before the experiment (10 hours fast). ¹¹¹In-labeled GRDA was taken orally with a glass of water (240 ml) immediately after a standardized low calorie meal (˜240 kcal) labeled with ^{99m}Tc-tin colloid. ^{99m}Tc-tin colloid and ¹¹¹InCl₃ were provided by the West of Scotland Radionuclide Dispensary, Glasgow, UK.

γ-Scintigraphic imaging was performed with the subject in a standing position. Anterior and posterior static acquisitions of 25 second duration were collected before dosing, immediately after dosing then every 15 minutes for 5 hours; then every 30 minutes until the ORDA had emptied from the subject's stomach until fourteen hours post dose. Subsequent images were taken at 18 and 24 hours post dose.

Subjects were given a light lunch at five hours post-dose, a snack at 7 hours post dose and an evening meal at 10 hours post-dose. Breakfast was provided at the end of the study period (24 hours post dose). De-caffeinated fluids were allowed ad libitum after lunch

### Imaging:

The γ-scintigraphy was performed using Siemens E-cam fitted with a general purpose collimator. The image analysis was conducted using the Weblink Image Analysis programme.

### Results

The positioning and movement of the radiolabeled GRDA in the GI tract may be observed by γ-scintigraphy simultaneously with the labeled food ingested by the subject by observing, in two channels the energy of the two elements used.

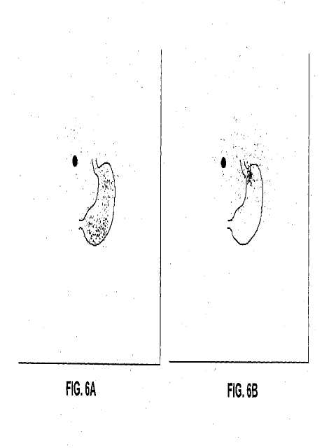

Specifically, FIG. 6A is a γ-scintigraphy image showing the ^{99m}Tc-labelled food in the subject's stomach immediately after eating. FIG. 6B is a γ-scintigraphy image showing the ¹¹¹In-labeled GRDA in the stomach immediately after dosing.

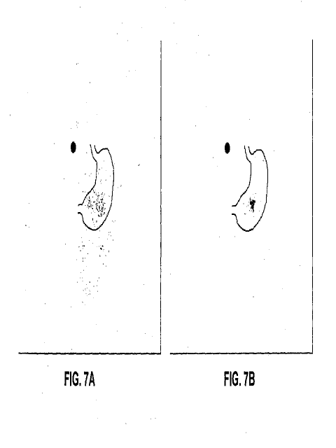

FIG. 7A is a γ-scintigraphy image showing the T₅₀ meal evacuation (i.e. when 50% of the food content has moved out of the stomach) at 1.5 h after eating, while the ¹¹¹In-labeled GRDA is retained in the stomach (FIG. 7B).

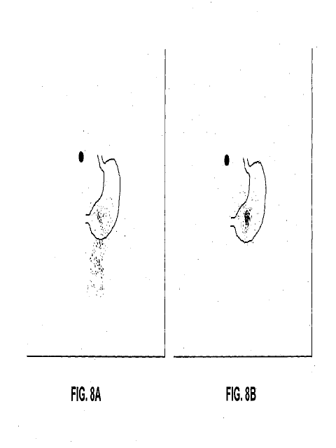

FIG. 8A is a γ-scintigraphy image showing the T90 meal evacuation at 2.0 h after eating, while the ¹¹¹In-labeled GRDA is retained in the stomach (FIG. 8B).

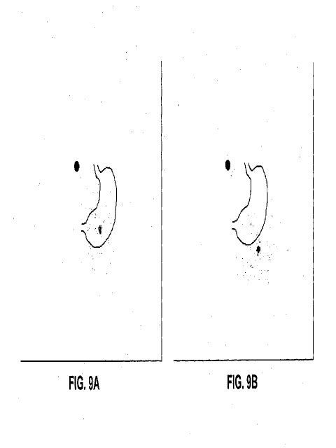

FIG. 9A is a γ-scintigraphy image of the ¹¹¹In-labeled GRDA in the stomach at 5.0 h, while FIG. 9B is a γ-scintigraphy image of the ¹¹¹In-labeled GRDA after 5.5 hours, showing the exit of the GRDA from the stomach.

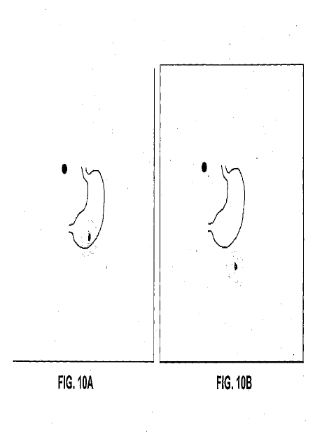

Finally, FIGS. 10A and 10B are the respective γ-scintigraphy images of the control tablet at 3.75 and 4 h showing that the control table is released from the stomach after 4 hours.

Overall, the results presented in the γ-scintigraphy images indicate that the GRDA does not interfere with the normal motility of the stomach, as there is no interference with food evacuation time. Food evacuation times are summarized in Table 4, while Table 5 summarizes the times the GRDA was retained in the stomach in 8 human subjects.

TABLE 4
GRDA vs. control tablet at T₅₀ and T₉₀

| | |
|---|---|
| T50 | T90 |

| Subject | GRDA | tablet | GRDA | Tablet |
|---|---|---|---|---|
| 1 | 0.40 | 0.34 | 1.21 | 1.08 |
| 2 | 1.37 | 1.52 | 1.98 | 2.88 |
| 3 | 0.57 | 0.47 | 0.91 | 1.21 |
| 4 | 0.68 | 0.54 | 1.41 | 1.14 |
| 5 | 1.17 | 0.84 | 1.81 | 1.49 |
| 6 | 0.43 | 0.54 | 1.21 | 1.16 |
| 7 | 0.93 | 0.70 | 1.22 | 1.72 |
| 8 | 0.64 | 0.69 | 1.65 | 1.41 |
| Mean | 0.77 | 0.71 | 1.43 | 1.51 |
| Median | 0.66 | 0.62 | 1.32 | 1.31 |
| SD* | 0.35 | 0.36 | 0.36 | 0.59 |
| Minimum | 0.40 | 0.34 | 0.91 | 1.08 |
| Maximum | 1.37 | 1.52 | 1.98 | 2.88 |

*SD—standard deviation

TABLE 5
GRDA retention in the stomach.

| Subject | GRDA | Tablet | GRDA | Tablet |
|---|---|---|---|---|
| 1 | 24 | 5 | 18 | 4.75 |
| 2 | 5.5 | 4 | 5 | 3.75 |
| 3 | 24 | 4.5 | 18 | 4.15 |
| 4 | 5.5 | 3.5 | 5 | 4.75 |
| 5 | 4 | 2.5 | 3.75 | 3.5 |
| 6 | 24 | 4 | 18 | 3.75 |
| 7 | 24 | 3.75 | 18 | 3.5 |
| 8 | 3.5 | 2 | 3.25 | 3 |
| Mean | 14.3 | 3.7 | 11.1 | 3.9 |
| Median | 14.8 | 3.9 | 11.5 | 3.8 |
| SD* | 10.38 | 0.99 | 7.37 | 0.62 |
| Minimum | 3.5 | 2 | 3.25 | 3 |
| Maximum | 24 | 5 | 18 | 4.75 |

*SD—standard deviation

1-52. (canceled)

53. A gastro-retentive diagnostic assembly (GRDA) for use in determining a condition of a subject's GI tract, comprising a folded single or multi-layered device comprising a diagnostic utility, the device prior to folding being essentially planar, and included in a delivery system for oral intake, the delivery system being adapted to release the device once in said subject's stomach, whereupon release said device unfolds into an unfolded shape that results in the retention of the device in the stomach.

54. The GRDA of claim 53, wherein said diagnostic utility comprises a contrasting agent.

55. The GRDA of claim 54, wherein the contrasting agent is a contrasting agent for X-ray, ultrasound, γ-scintigraphy or MRI imaging.

56. The GRDA of claim 54, wherein said contrasting agent is retained in the device once unfolded for a period of time permitting imaging of the stomach.

57. The GRDA of claim 56, wherein the contrasting agent is released from the device at a rate so as to permit imaging of the device throughout a substantial portion of time of the device's retention in the stomach.

58. The GRDA of claim 53, wherein the delivery system is selected from a capsule containing the folded device, a tube surrounding the folded device, a polymeric coating, a polymer or gel matrix embedding the folded device.

59. The GRDA of claim 53, wherein the device comprises a single layer comprising said diagnostic utility.

60. The GRDA of claim 59, wherein said diagnostic utility is adsorbed onto, or embedded in said single layer or is absorbed into a carrier that is attached to said single layer.

61. The GRDA of claim 53, wherein the device comprises two layers sandwiching said contrasting agent between them.

62. The GRDA of claim 53, wherein said single or multi-layered device comprises a matrix, and said diagnostic utility is adsorbed to, embedded in or sandwiched between matrix layers, or is absorbed into a carrier that is attached to the layers, or any combination thereof.

63. The GRDA of claim 53, wherein said device comprises a polymeric composition for maintaining a configuration of the device when unfolded that provides for said retention of the device in the stomach.

64. The GRDA of claim 63, wherein said polymeric composition is attached to said matrix or is integrally formed therewith.

65. The GRDA of claim 53, where the device unfolds into a generally planar configuration.

66. The GRDA of claim 53, wherein folding to yield said folded device is by one or more of folding about fold lines, rolling, bending twisting, winding or crimping.

67. The GRDA of claim 53, wherein said diagnostic utility is associated with a vector for delivery of the diagnostic utility to the stomach's lumen, the diagnostic utility associated with the vector being releasable from the device when the device is in an unfolded state.

68. The GRDA of claim 53, wherein said device is folded in an accordion-like or fan-like configuration.

69. The GRDA of claim 53, wherein said delivery system is a hard gelatin capsule.

70. A method of determining a condition of a subject's GI tract comprising:
administering to a subject with a GRDA comprising a folded single or multi-layered device comprising a diagnostic utility, the device prior to folding being essentially planar, and being included within a delivery system for oral intake, the delivery system adapted to release the device once in the subject's stomach, whereupon release, said device unfolds into an unfolded shape that results in the retention of the device in the stomach; and
retrieving data indicative of a condition of the subject's GI tract.

71. The method of claim 70, wherein said diagnostic utility comprises a contrasting agent.

72. The method of claim 70, comprising imaging of said subject's GI tract.

73. The method of claim 70, wherein said imaging of the GI tract comprises X-ray, ultrasound, γ-scintigraphy or MRI imaging.

74. The method of claim 71, wherein said contrasting agent is retained in the device once unfolded for a period of time permitting imaging of the GI tract.

75. The method of claim 71, wherein the contrasting agent is released from the device at a rate so as to permit imaging of the device throughout a substantial portion of time of the device's retention in the stomach.

76. The method of claim 70, wherein the device comprises two layers sandwiching said diagnostic utility between them.

77. The method of claim 70, wherein said single or multi-layered device comprises is a matrix and said diagnostic utility is adsorbed to, embedded in or sandwiched between the layers.

78. The method of claim 70, wherein upon delivery to the subject said device is folded in an accordion-like or fan-like configuration in said delivery system.

79. The method of claim 70, wherein said delivery system is a hard gelatin capsule.

80. The method of claim 70, wherein said data indicative of said condition of the GI tract is retrieved when the single or multi-layered device is in a generally planar shape.

81. The method of claim 70, wherein said data is retrieved within a time window of at least 48 hours post administration of the GRDA to the subject.

82. The method of claim 81, wherein said time window is of at least 18 hours post administration.

83. The method of claim 82, wherein said time window is of at least 10 hours post administration.

84. The method of claim 82, wherein said time window is of at least 5 hours post administration.

85. The method of claim 70, wherein said diagnostic utility comprises a contrasting agent and said method comprises capturing one or more images during said time window.

86. The method of claim 70, for determining a pathological condition in a subject's GI tract.

87. The method of claim 86, wherein said pathological condition is a condition of the stomach.

88. The method of claim 87, wherein said condition is a condition of the stomach selected from gastroparesis, gastritis, gastroenteritis, gastric ulcer and gastric cancer

89. The method of claim 86, wherein said pathological condition is selected from irritable bowel syndrome, GI bleeding, GI portal hypertension, colitis, diverticulosis, colon polyps, GI cancer, carcinoid, inflammatory bowel disease (IBD), GI obstructions and metabolic diseases associated with excess or deficient secretion of gut hormones.

90. The method of claim 70, for monitoring a change in a pathological condition in a subject's GI tract.

91. The method of claim 90, comprising sequential administrations of a GRDA to a subject, each administration followed by retrieval of data indicative of the condition of the subject's GI tract.

92. Use of a generally planar single or multi-layered device comprising a diagnostic utility for the preparation of a GRDA for oral intake, the GRDA comprising said device in a folded configuration and included in a delivery system, the delivery system being adapted to release the single or multi-layered device once in the stomach whereupon release, said device unfolds into an unfolded shape that results in the retention of the device in the stomach.

93. The use of claim 92, wherein said GRDA is for determining a pathological condition in a subject's GI tract or for monitoring a change in a pathological condition in a subject's GI tract during or after providing said subject with a treatment for said pathological condition.

94. A method for preparing a GRDA for use in determining a condition of a subject's GI tract, the method comprises: (i) providing an unfolded and essentially planar single or multi-layered device comprising a diagnostic utility; (ii) folding said device; and (iii) introducing or combining the folded device with a delivery system, such that when in the stomach it is released from the delivery system, whereupon release it unfolds into an unfolded shape that results in the retention of the device in the stomach.
